(19) United States
(12) Patent Application Publication
(10) Pub. No.: US 2004/0241210 A1
(43) Pub. Date: Dec. 2, 2004

(54) Method for applying sunless tanning composition to the human body using a tight fitting garment as the applicator

(76) Inventor: Kelli K. Jenkel, Grand Junction, CO (US)

Correspondence Address: Kelli K. Jenkel, 234 Red Mesa Hts, Grand Junction, CO, 81503, US

(21) Appl. No.: 10/446,421

(22) Filed: May 28, 2003

(51) Int. Cl. A61K 9/70

(52) U.S. Cl. 424/443

(57) ABSTRACT

A method of sunless tanning (24) for providing an evenly dispersed darkening (20) of the human skin where a tight-fitting garment (14) is the means of application for a sunless tanning composition (10) which is impregnated into garment (12) then worn for a period of time (16) by a human being. The effect of the tan is evaluated (18) after the period of time (16) for sufficient darkening (20). Method is repeated until darkening (20) is achieved. A decision (22) is made to convert garment (14) into conventional tight-fitting garment (12) by laundering.

### BACKGROUND

This invention relates to sunless tanning, specifically to the application of a sunless tanning composition having means that allow users to apply the sunless tanning agent Dihydroxyacetone (DHA) by wearing a tight-fitting garment impregnated with the sunless tanning composition through the normal course of a workday or evening activities.

The current methods used to apply sunless tanning composition can be time consuming, provide uneven results, and are expensive. These methods for the application of the sunless tanning composition are categorized in two divisions by which means the sunless tanning composition is either a manual application of a composition containing Dihydroxyacetone (DHA) or an atomized application of said composition. The first, the manual application, is performed by applying the mixture in an emulsion form directly to the skin by means of the bare hand, a latex glove, foam brush, or spray bottle. The second, an atomized application, is performed in a commercial application by spraying the mixture on the body in a closed shower-like apparatus.

Both U.S. Pat. No. 5,514,437 to Tanner (1996) and U.S. Pat. No. 5,922,333 to Laughlin (1999) demonstrate the methods for the application of sunless tanning compositions to the human body. Tanner invents a sunless tanning composition with improved stability which implies application in emulsion, manually. Laughlin offers several embodiments of sunless tanning compositions which are in turn atomized and applied to the entire human body with means of spray nozzles. U.S. Pat. No. 5,664,593 to McClain (1997) describes an apparatus for applying suntanning lotion in an atomized mist to portions of the human body.

Both the Tanner and the Laughlin method require the user to make extraordinary efforts to achieve skin darkening. The usual commercial sunless tanning composition derived from Tanner's sunless tanning composition is manually applied. The manual application of sunless tanning composition relies heavily on the user's ability to allocate up to three hours of intense activity before a long period of virtually no activity to achieve the desired result of an evenly shaded skin tone. During the application period the user must first shower and exfoliate the skin, provide sacrificial clothing for use both before and after the application, provide applicators such as foam paint brushes for hard to reach areas, and provide additional lotions and moisturizers for the protection of sensitive skin areas. The user must then remain nude or nearly nude for up to an hour after the application while the sunless tanning composition drys in order to prevent streaking and damage to clothing. A sunless tan achieved in this manner will produce an effective darkening of the skin for approximately four days.

The atomizing method of Laughlin provides an improved method for achieving an evenly darkened skin coloration. However, the cost of the equipment is beyond the reach of most individual users and is commonly sold only to tanning salons. The user of the atomized sunless tanning composition must also guard against stained clothing. Furthermore, the user must either purchase expensive apparatus or share publicly used facilities, and plan to reuse the service every 4 to 5 days.

In conclusion, insofar as I am aware, no method of applying a sunless tanning composition to the human body using a tight fitting garment as the applicator is in use or in publication.

### SUMMARY

The invention, an improved method of applying sunless tanning composition to the human body by means of impregnating the fabric of a tight-fitting garment with said sunless tanning composition and then wearing said garment throughout the normal day or evening hours combines two commonly used items into one novel and useful method. An item selected from a group of garments such as panty hose, unitards, leotards, and undergarments when combined with said sunless tanning composition containing the sunless tanning agent Dihydroxyacetone can impart an evenly dispersed darkened tone to the skin it contacts, said darkening approximating the color achieved through normal ultra-violet sun tanning. Additionally, no extraordinary preparation is necessary to achieve these results, and the applicator is completely reusable. The art of sunless tanning is now over forty years old and the manufacture of tight-fitting garments is much older. The combination of these two items is therefore both novel and unobvious to the workers of the art in both the field of sunless tanning methods and the field of manufacturing tight-fitting garments.

Accordingly the following are objects and advantages of the invention. The method provides an improved means of using sunless tanning compositions. The invention will reduce the expense and increase the opportunity for the use of sunless tanning compositions, reduce the need for expensive atomizing equipment, offer ease-of-use improvements to the user, compliment the effort to conserve energy and natural resources by offering added-value to currently produced consumables, and expand the market for producers of both tight-fitting garments and sunless tanning products. Furthermore, the expanded use of sunless tanning compositions will reduce the overall incidence of certain cancers and cancer related expenses. Still further objects and advantages will become apparent to workers in the art from study of the following drawing and description.

### DRAWINGS

FIG. 1 is a flow chart representing the method of imparting darkened skin color through the use of this invention.

### DESCRIPTION

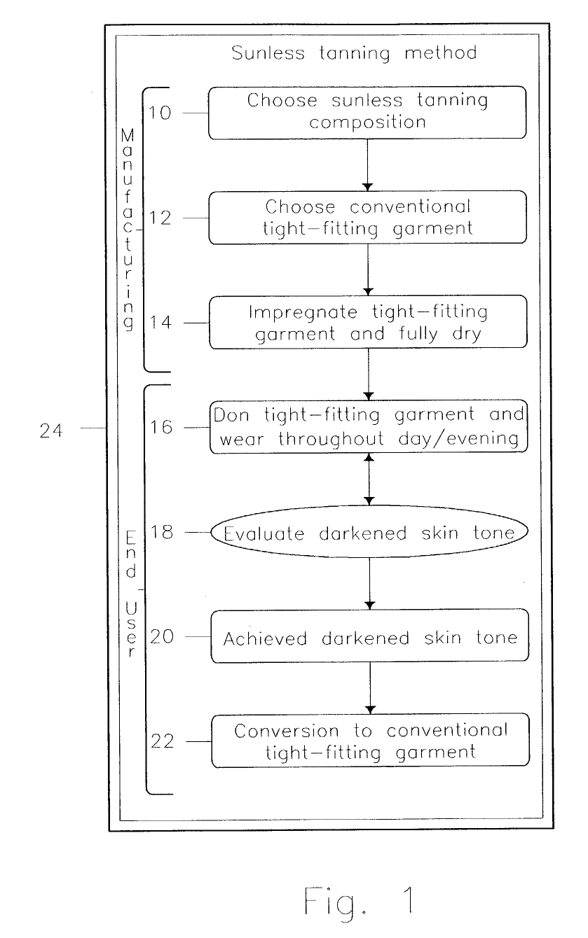

FIG. 1 is a flow chart representing the method of using a tight-fitting garment to apply sunless tanning composition to a skin surface of the human body. A selection 10 is made for which type of sunless tanning composition will be impregnated into a selection of garment 12 type. Garment 12 is impregnated with composition 10 and then allowed to fully dry. Garment 14 will retain the potential to impart an essential sunless tanning agent contained within the impregnating sunless tanning composition to the surface of skin. Garment 14 is donned and is worn 16 as a normally worn garment throughout the work day or evening hours. An evaluation 18 is made at the end of the workday or evening hours considering the achieved darkened skin tone 20. If darkening 20 is sufficient, evaluation 18 will be made again during each of the following four to five days. When darkening 20 is no longer deemed sufficient, Garment 14 is re-donned and worn 16 to add additional darkening to the skin it contacts. Garment 14 can be reused until it is deemed soiled, at which time it can be laundered and then converted 22 as a conventional tight-fitting garment 12 until the end of its useful life.

Reference Numerals

10 Choose sunless tanning composition

12 Choose conventional tight-fitting garment

14 Impregnate tight-fitting garment and fully dry

16 Don tight fitting garment and wear throughout the workday or evening hours

18 Evaluate darkened skin tone

20 Achieved darkened skin tone

22 Conversion to conventional tight-fitting garment

24 Sunless tanning method

Operation

In operation one uses this method to gain a sunless tan without a dangerous exposure to ultra-violet radiation. The user avoids the expense of either purchasing an atomization apparatus or making regular service payments to tanning salons or health clubs which supply the atomization apparatus. Alternatively, the user avoids the time consuming and unpredictable results expected when manually applying sunless tanning compositions. When the means of this invention is implemented:

(1) The user follows a normal routine in preparation for either a workday or evening activities.

(2) The user dons a previously prepared garment 14 in anticipation of obtaining a sunless tan, perceived as a darkened skin tone 20, on those areas of the skin which contact the prepared garment 14.

(3) The user evaluates the effect imparted by said garment 14 at the end

(3) The user evaluates the effect imparted by said garment 14 at the end of the workday or evening activities 16 for sufficient darkening 20 or for conversion 22 to a normal garment 12 through laundering.

When the user wishes to increase the darkening 20 effect, it is necessary to wear the garment 16 for longer intervals or more frequently.

1. In a method of sunless tanning, a means comprising the combination of:
a tight-fitting garment which will provide constant contact with human skin, and
a sunless tanning composition which is impregnated into said tight-fitting garment, and
a period of time during which the garment impregnated with said composition is worn by a human,
whereby during said period of time, the user will by means of wearing said garment obtain a darkened skin tone on those areas of the skin contacted by said garment.
